(19) United States
(12) Patent Application Publication
(10) Pub. No.: US 2023/0355692 A1
(43) Pub. Date: Nov. 9, 2023

(54) COMPOSITIONS COMPRISING A VEGETABLE CHONDROITIN OR AN ANALOGUE THEREOF AND THE USE THEREOF IN THE TREATMENT OF DISORDERS OF THE MUCOUS MEMBRANE OF THE ORAL, PHARYNGO-LARYNGEAL AND/OR GASTRO-OESOPHAGEAL TRACT

(71) Applicant: SOFAR SWISS SA, Lugano (CH)

(72) Inventor: Andrea Biffi, Lugano (CH)

(73) Assignee: SOFAR SWISS SA, Lugano (CH)

(21) Appl. No.: 18/246,222

(22) PCT Filed: Sep. 23, 2021
(86) PCT No.: PCT/IB2021/058677

§ 371 Date: Mar. 22, 2023

(30) Foreign Application Priority Data
Sep. 23, 2020 (IT) 102020000022477

(51) Int. Cl. A61K 36/03; A61K 31/728; A61K 36/05; A61K 33/08; A61K 33/12; A61K 9/00; A61P 1/04

(52) U.S. Cl. CPC A61K 36/03; A61K 31/728; A61K 36/05; A61K 33/08; A61K 33/12; A61K 9/0053; A61P 1/04

(57) ABSTRACT

The present invention relates to mixtures or compositions comprising a chondroitin sulfate of plant origin or an analogue thereof (algae extract) and to the use thereof in the preventive or curative treatment of diseases and symptoms caused by gastric reflux in the gastroesophageal tract or in extra-oesophageal regions, and ulcers, lacerations and/or inflammations of the mucous membranes or the lining tissues of the various anatomical regions present in the tract from the stomach to the oral cavity, also including the upper respiratory tract.

The present invention relates to mixtures or compositions comprising a chondroitin sulfate of plant origin or an analogue thereof, wherein said analogue comprises or, alternatively, consists of an algae extract and, optionally, a hyaluronic acid. Furthermore, the present invention relates to said mixtures or said compositions comprising a chondroitin of plant origin or said analogue thereof for use in the preventive or curative treatment of diseases and/or symptoms of the oral mucous membrane, the mucous membrane of the laryngopharyngeal tract and/or the mucous membrane of the gastro-oesophageal tract, such as for example diseases and/or symptoms caused by gastric reflux in the gastroesophageal tract or in extra-oesophageal regions, and ulcers, lacerations and/or inflammations caused in the mucous membrane or in the lining tissues of the various anatomical regions present in the tract from the stomach to the oral cavity, also including the upper respiratory tract.

Damage to or inflammation of the mucous membranes of the oral tract and of the upper respiratory tract, the laryngopharyngeal and/or gastro-oesophageal tract are a widespread problem in the population which, besides deteriorating the quality of life and causing difficulty in the intake of food and beverages, they can result in permanent, high-severity damage.

Located in the gastric mucosa, parietal cells are responsible for the secretion of hydrochloric acid in the stomach. Chemical and physical stimuli induce the release of numerous mediators which finely regulate this secretion. However, in some cases there is an imbalance in these regulations, resulting in acid hypersecretion. Furthermore, a malfunction of the lower oesophageal sphincter leads to the backflow of gastric acid secretion and/or biliary and/or combined secretion from the stomach to the oesophagus and/or to extra-oesophageal regions. Said acid hypersecretion and/or said backflow of gastric acid secretion and/or biliary and/or combined secretion may lead to pathological conditions such as gastroesophageal reflux disease, pharyngo-laryngeal (or extra-oesophageal) reflux disease and/or the formation of ulcers on the mucous membrane of the upper tract of the digestive tract up to the oral cavity, also including the upper respiratory tract.

Gastroesophageal reflux disease (GERD) is a clinical condition characterised by massive backflow of the gastro-duodenal content into the oesophagus. The highly acidic gastric content and bile contained therein at contact with the oesophageal mucosa lead to inflammatory conditions which may result in pyrosis or oesophageal ulcers. The characteristic symptoms of this disorder can be divided into typical (e.g. acid regurgitation, heartburn), atypical (e.g. feeling of gastric fullness, epigastric pain, dyspepsia, nausea) and extra-oesophageal (e.g. chronic cough, bronchospasm, laryngospasm, raucousness, globus pharyngis, dysphonia, dysphagia, excessive throat clearing, sore or burning throat, inflamed throat, postnasal drip, laryngitis, pharyngitis, and/or other symptoms of the upper respiratory tract).

Laryngopharyngeal reflux disease (LPR) or extra-oesophageal reflux is a clinical condition characterised by the backflow of the gastro-duodenal content from the stomach to the extra-oesophageal regions such as the upper respiratory tract (the nasal cavity, the paranasal sinuses, the oral cavity, the pharynx, the epiglottis and the larynx).

Acid hypersecretion, gastroesophageal, pharyngo-laryngeal or extra-oesophageal reflux, ulcers in gastric and oesophageal mucous membranes and other symptoms or disorders related thereto can be treated either through pharmacological therapy (e.g. H2 antihistamines; proton pump inhibitors (PPI), etc.) both through compounds which exert a mainly mechanical action (for example, floating raft or the formation of mucous membrane lining films).

The use of compositions comprising chondroitin sulfate for the prevention and/or treatment of inflammations, ulcers and/or lesions of the mucous membranes of the oral tract, laryngo-pharyngeal tract and/or gastro-oesophageal tract, is known in the art. However, the chondroitin or chondroitin sulfate used in products or medicaments available on the market is obtained through extraction from animal cartilage, such as for example, cartilage from cattle, pigs, chicken, sharks, fish or crustaceans. To date, the processes for the preparation of compounds intended for human use (for example for nutritional, biomedical or pharmaceutical applications) obtained using an animal source as raw material face several limits and drawbacks, many criticisms linked to the safety of the compound, as well as criticisms of ethical, religious and moral nature. Besides the above, vegans and vegetarians remain categories of subjects excluded from the use of specific products, such as for example products or compositions for the prevention and/or treatment of inflammations, ulcers and/or lesions of the mucous membranes of the oral tract, of the upper respiratory tract, of the laryngo-pharyngeal tract and/or of the gastro-oesophageal tract, given that they contain a chondroitin or chondroitin sulfate of animal origin.

US 2019/262388 A1 illustrates a liquid composition for use in the treatment of gastroesophageal reflux, wherein said composition comprises a mixture comprising or, alternatively, consisting of a combination of an Aloe vera gel, hyaluronic acid and honey and, optionally, food or pharmaceutical grade additives and/or technological excipients.

US 2019/125664 A1 illustrates a chewable and/or oral-soluble tablet and/or a mouth-dissolvable tablet based on hyaluronic acid and chondroitin sulfate and/or salts thereof.

US 2006/040894 A1 illustrates compositions and devices which include hyaluronic acid and a compound that inhibits the degradation of hyaluronic acid, and methods for production and use thereof.

US 2015/209264 A1 describes a combination of pullulan or a derivative thereof with a polysaccharide selected from hyaluronic acid, a salt or derivative thereof, alginic acid, a salt or derivative thereof and a mixture thereof.

US 2017/049678 A1 illustrates a combination comprising a hyaluronic acid or a salt thereof and a sulfated polysaccharide whose molecular weight is comprised between 5 kDa and 25 kDa, that is in particular used for combatting signs of ageing of the skin or for the treatment and the healing of skin wounds.

US 2008/063682 A1 illustrates compositions and methods that involve the administration of agents useful for the treatment, prevention and inhibition of inflammatory diseases or fibrous adhesions using sulfated fucans and, if desired, one or more other anti-inflammatory or anti-fibrous adhesion agents.

EP 3 240 550 A1 illustrates a combination of hyaluronic acid, chondroitin sulfate and magaldrate for the prevention or the treatment of gastric hyperacidity and gastroesophageal reflux, and related diseases. TZIVELEKA LETO-AIKATERINI ET AL: “Ulvan, a bioactive marine sulphated polysaccharide as a key constituent of hybrid biomaterials: A review”, CARBOHYDRATE POLYMERS, vol. 218, pages 355-370, is a study which contains general information on the chemical structure and variability, extraction processes, physico-chemical properties and biological activities of ulvan.

Therefore, in the field of pharmaceuticals, compositions for medical devices, nutraceuticals, foods for special medical purposes (FSMPs), dietary supplements or foodstuffs, there is felt the need on the part of market operators to have products or medicaments available to all categories of consumers, including vegans, vegetarians, subjects suffering from allergies and subjects who, for religious or ideological reasons, cannot consume products or medicaments comprising compounds or ingredients of animal origin. Furthermore, the need is felt to produce products or compositions containing only ingredients of plant origin that are cost-effective, easy to prepare and stable over time.

The technical problem addressed and solved by the present invention lies in providing compositions that are effective and free of side effects and that can be administered to any category of subjects for use in a method for the preventive or curative treatment of diseases and/or symptoms of the oral mucous membrane and the upper respiratory tract, the mucous membrane of the laryngopharyngeal tract and the mucous membrane of the gastro-oesophageal tract, such as for example diseases and/or symptoms caused by gastric reflux in the gastroesophageal tract or in extra-oesophageal regions, and ulcers, lacerations and/or inflammations caused in the mucous membrane or in the lining tissues of the various anatomical regions present in the tract from the stomach to the oral cavity, also including the upper respiratory tract.

Furthermore, the technical problem addressed and solved by the present invention lies in providing compositions effective in the aforementioned treatments which have an excellent pharmacokinetic profile when administered to a subject through the oral route, that is an excellent intestinal permeability and a high blood bioavailability of the entire composition or at least of an active ingredient.

In order to overcome said technical problems, following an intense research and development phase, the Applicant provides mixtures or compositions comprising a chondroitin or chondroitin sulfate of plant origin or an analogue thereof, wherein said analogue comprises or, alternatively, consists of an extract of at least one alga and, optionally, a hyaluronic acid, wherein said at least one alga is an alga belonging to the species Ulva lactuca (L.) and/or an alga belonging to the genus Fucus (L.), preferably to the species Fucus vesiculosus, as reported in the present description and in the attached claims.

Said mixtures or compositions of the invention comprising a chondroitin or a chondroitin sulfate of plant origin or said analogue thereof, when administered to a subject in need are able to exert—in a lasting and effective manner—a mechanical protection of the lining of the mucous membranes or of the tissues present in the tract that goes from the stomach to the oral cavity, also including the upper respiratory tract as well as an anti-inflammatory action and an action for the re-epithelization and/or cicatrisation of said mucous membranes or tissues. As a result, said mixtures or compositions of the invention comprising a chondroitin or a chondroitin sulfate of plant origin or said analogue thereof are capable of preventing and/or treating diseases and/or symptoms of the mucous membrane of the oral tract and the upper respiratory tract, of the laryngo-pharyngeal tract and/or the gastro-oesophageal tract in an effective manner and without side effects. Furthermore, the mixtures and compositions of the invention comprising said chondroitin or chondroitin sulfate or an analogue thereof of plant origin, can be used by all categories of subjects, including those subjects who cannot or do not want to consume products fully or partly derived from animal sources.

Lastly, the mixtures or the compositions of the present invention are easy to prepare and cost-effective to produce.

These and other objects which will be apparent from the detailed description that follows are achieved by the mixtures and the compositions the present invention thanks to the technical characteristics present in description and in the attached claims.

A first aspect of the present invention relates to a mixture (in short, mixture of the invention) comprising, or alternatively, consisting of: (a.1) a low molecular weight chondroitin or chondroitin sulfate of plant origin or a salt thereof, preferably a sodium chondroitin sulfate, or (a.2) an analogue of a chondroitin or vegetable chondroitin sulfate, such as a mixture comprising or, alternatively, consisting of an extract of at least one alga and, optionally, a hyaluronic acid, wherein said at least one alga is an alga belonging to the species Ulva lactuca (L.) and/or an alga belonging to the genus Fucus (L.), preferably to the species Fucus vesiculosus.

A second aspect of the present invention relates to a composition (in short, composition of the invention) comprising said mixture of the invention and at least one acceptable pharmaceutical or food grade additive and/or excipient.

A third aspect of the present invention relates to said mixture or said composition of the invention, comprising a chondroitin sulfate of plant origin or said analogue thereof (i.e. extract of at least one alga and a hyaluronic acid), for use as medicament, preferably for use in a method for the preventive or curative treatment of diseases and/or symptoms of the oral mucous membrane and of the upper respiratory tract, of the mucous membrane of the laryngopharyngeal tract and of the mucous membrane of the gastro-oesophageal tract, as defined in the context of the present description.

A fourth aspect of the present invention relates to the non-medical use of said chondroitin or chondroitin sulfate of plant origin or said analogue thereof (i.e. extract of at least one alga and, optionally, a hyaluronic acid) for the preparation of cosmetic compounds and/or as an additive or excipient in the preparation of pharmaceuticals, medical devices, nutraceuticals, foods for special medical purposes (FSMPs), dietary supplements or foodstuffs.

### FIGURES

FIG. 1 represents the Lucifer Yellow test results after 15 minutes+2 hours of treatment and application of caffeine.

### DETAILED DESCRIPTION OF THE INVENTION

A first aspect of the present invention relates to a mixture (in short, mixture of the invention) comprising, or alternatively, consisting of:
(a.1) a chondroitin or a chondroitin sulfate of plant origin or a salt thereof, preferably a sodium chondroitin sulfate, having an average molecular weight comprised from about 1 kDa to 250 kDa, preferably comprised from 1 kDa to less than or equal to 50 kDa (≤50 kDa), more preferably from about 1 kDa to about kDa (e.g. from 5 kDa to 15 kDa), or
(a.2) an analogue of a chondroitin or vegetable chondroitin sulfate, such as a compound comprising a mixture comprising or, alternatively, consisting of an extract of at least one alga and, optionally, a hyaluronic acid or a salt thereof (hyaluronate), wherein said at least one alga comprises or, alternatively, consists of an alga belonging to the species Ulva lactuca (L.) and/or an alga belonging to the genus Fucus (L.) (preferably belonging to the species Fucus vesiculosus).

The expressions “vegetable chondroitin sulfate” and “chondroitin sulfate of plant origin” are synonyms and identify a chondroitin sulfate or a salt thereof derived entirely from a plant source and which do not comprise animal derivatives.

In the context of the present invention the expression “chondroitin sulfate” is used to indicate both chondroitin sulfate as such and an acceptable pharmaceutical or food grade salt thereof, preferably sodium chondroitin sulfate (mono- or di-sodium).

In the context of the present invention, the expressions “chondroitin sulfate” and “chondroitin” may be used as synonyms and interchangeably.

In the context of the present invention, said mixture comprising or, alternatively, consisting of an extract of at least one alga and, optionally, a hyaluronic acid or a salt thereof (hyaluronate) may be referred to as “analogue of a chondroitin sulfate” or “vegetable substance”, for the sake of simplicity.

Chondroitin sulfate is a sulfated glycosaminoglycan (in short, GAG), such as a polysaccharide of variable length containing two alternating monosaccharides: N-acetyl-D-galactosamine and D-glucuronic acid. Chondroitin sulfate can be used in the context of the present invention in the form of chondroitin sulfate salt, such as for example, sodium, potassium, calcium, magnesium or aluminium salt, preferably sodium chondroitin sulfate.

Said (a.1) chondroitin sulfate or a salt thereof (preferably sodium chondroitin sulfate), used in the context of the present invention consists of a chondroitin sulfate obtained by extracting and/or fermenting from a plant source (for example at least one plant and/or one fungus and/or one alga) having a low average molecular weight, a homogeneous profile and a low polydispersion, as described in the present invention.

Said (a.1) vegetable chondroitin sulfate or a salt thereof (for example a sodium chondroitin sulfate) used in the context of the present invention have an average molecular weight comprised from about 1 kDa to 250 kDa (for example 100 kDa, 150 kDa or 200 kDa), preferably from about 1 kDa to less than or equal to 50 kDa (for example, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa, 20 kDa, 25 kDa, 30 kDa, 35 kDa, 40 kDa or 50 kDa), preferably from about 1 kDa to about 20 kDa, more preferably from about 5 kDa to about 15 kDa (1000 Da=1 kDa, Da=Dalton). Said (a.1) low molecular weight vegetable chondroitin sulfate (for example about 1-50 KDa or about 1-20 KDa) exhibits properties of greater permeation at the intestinal level and greater blood bioavailability when administered to a subject, with respect to a chondroitin sulfate having a higher molecular weight (for example >50 KDa or >100 kDa or 250-1000 KDa) such as for example, a chondroitin of animal origin (e.g. chicken).

Preferably, the chondroitin sulfate of plant origin or a salt thereof (for example sodium chondroitin sulfate) used in the context of the present invention have a charge density comprised from about 0.70 to about 1.50 (for example 0.80. 0.85, 0.87, 0.90. 0.92, 0.94, 0.96, 1.10, 1.30 or 1.40), preferably comprised from about 0.75 to about 1.20, more preferably comprised from about 0.80 to about 0.99.

The vegetable chondroitin sulfate or a salt thereof (for example sodium chondroitin sulfate) used in the context of the present invention preferably comprise a by weight percentage of 6-chondroitin sulfate comprised from 50% to 99.5% (for example about 55%, 60%, 65%, 70%, 75%, 80%, 85%, 90%, 95% or 98%), preferably from 75% to 90% or 95%, and a by weight percentage of 4-chondroitin sulfate comprised from about 0.01% to about 10% (for example about 0.05%, 0.1%, 0.2%, 0.3%, 0.4% 0.5%, 1%, 2%, 3%, 4%, 5%, 6%, 7%, 8%, or 9%), preferably from 0.1% to 5% or 10%, with respect to the total of disaccharides contained in the chondroitin sulfate; said percentages (%) being determined, for example, by means of HPLC.

Said vegetable chondroitin sulfate or a salt thereof, further comprises low percentages by weight of chondroitin sulfate wherein the group SO₃H or SO₃⁻— is in position 4,6-CS and/or 2,6-CS and/or 2,4-CS, wherein for example said low percentages are comprised from 0.01% to 20% (for example about 0.1%, 0.5%, 1%, 2%, 4%, 5%, 8%, 10% or 15%), with respect to the total of disaccharides contained in said vegetable chondroitin sulfate.

An embodiment of said (a.1) chondroitin sulfate of plant origin is the sodium chondroitin sulfate having an average molecular weight from about 7 kDa to about 12 kDa (about 9 kDa), a protein content ≤0.5% (about 0.1%), a content of 6-chondroitin sulfate from about 75% to about 90% and 4-chondroitin sulfate in a percentage by weight comprised from 0.1% to about 5% (% by weight with respect to the total of disaccharides contained in chondroitin sulfate).

In the context of the present invention, said (a.2) composition comprising a mixture comprising or, alternatively, consisting of an extract of at least one alga (Ulva lactuca and/or Fucus) and, optionally, a hyaluronic acid, is defined as an “analogue of a vegetable chondroitin sulfate” (or “analogue of a vegetable chondroitin”) given that it is a composition or a mixture that mimics a chondroitin sulphate (or chondroitin), thanks to the presence—in said mixture (a.2)—of mucopolysaccharides, with which the algae Ulva lactuca and/or Fucus are rich, comprising glucuronic acid and N-acetylglucosamine.

Ulva lactuca (or sea lettuce) is a species of green seaweed of the class Ulvophyceae, order Ulvales, family Ulvaceae, genus Ulva.

Fucus is a genus of seaweed belonging to the class Phaeophyceae, order Fucales, family Fucaceae. There are various species belonging to the genus Fucus; in the context of the present invention the alga of the genus Fucus preferably belongs to the species Fucus vesiculosus.

N-acetylglucosamine is a modified monosaccharide having formula C8H15NO6 (N-acetylated derivative of 2-glucosamine), IUPAC name 2-acetylamino-2-deoxy-D-glucose (example of CAS No. 7512-17-6). Glucuronic acid (D-glucuronic acid, molecular formula C6H10O7) is an organic compound belonging to the category of alduronic acids and it derives from the oxidation of the primary alcohol group (OH bound to C-6) of D-glucose to a carboxylic group. Mucopolysaccharides (known as GAGs) are glycosaminoglycans whose long “unbranched” chains consisting of disaccharide units continue to repeat in determined order alternating an amino saccharide, that is a monosaccharide containing an amino functional group (—NH₂) instead of a hydroxyl functional group (—OH).

Said extract (or fermentation product) of at least one alga, wherein said at least one alga comprises or, alternatively, consists of Ulva lactuca and/or Fucus (preferably Fucus vesiculosus), is obtained by means of the methods and equipment suitable for the extraction of seaweed known to the person skilled in the art (for example extraction comprising a fermentation step). Said extract of Ulva lactuca and of Fucus may be obtained starting from a mixture of Ulva lactuca and of Fucus or by separately extracting Ulva lactuca and Fucus and combining the two extracts obtained.

Said (a.2) analogue of a vegetable chondroitin sulfate, such as a composition comprising a mixture, comprising or, alternatively, consisting of an extract of at least one alga (Ulva lactuca and/or Fucus) and, optionally, a hyaluronic acid (or a salt thereof) and at least one additive and/or excipient (preferably maltodextrin or wheat maltodextrin), is a product that does not comprise derivatives of animal origin, and advantageously it is “allergen free”. Said extract of Ulva lactuca and/or said extract of Fucus are rich with mucopolysaccharides (for example they comprise mucopolysaccharides in a percentage by weight from about 10% to about 95%, with respect to the total weight of the extract, such as about 20%, 30%, 40%, 50%, 60%, 70%, 80% or 90%).

Besides an extract of at least one alga (Ulva lactuca and/or Fucus), said (a.2) analogue of a vegetable chondroitin sulfate may further comprise a hyaluronic acid or a salt thereof, preferably a sodium hyaluronate, having an average molecular weight comprised from about 1 kDa to about 2000 kDa (for example about 100 kDa, 200 kDa, 300 kDa, 400 kDa, 500 kDa, 600 kDa, 700 kDa, 800 kDa, 900 kDa, 1000 kDa, 1200 kDa, or 1500 kDa).

An embodiment of said (a.2) analogue of a vegetable chondroitin sulfate that can be used in the context of the present invention is a compound comprising or, alternatively, consisting of an extract of Ulva lactuca and Fucus vesiculosus and, furthermore, wheat maltodextrin and, optionally, sodium hyaluronate, wherein said compound does not comprise derivatives of animal origin and it is “allergen free” having the following characteristics: powder form, density (bulk density): 0.4-0-6 g/ml, particle size: >95% through 30# (US sieve analysis), As, Pb, Cd, Hg amount according to the standard EP 7.0 (As <1 ppm, Pb <3 ppm, Cd <1 ppm, Hg <0.1 ppm), bacteria TVC <3.0×10³ CFU/g, yeasts <3.0×10³ CFU/g; solubility in water: about 2 g/L, contains only products permitted by EU legislation for food ingredients; and wherein said compound comprises—expressed as percentages by weight—an extract of Ulva lactuca from about 25% to 50% and an extract of Fucus vesiculosus from about 25% to 50%, maltodextrin from about 5% to 30%, and, optionally, sodium hyaluronate from about 1% to about 15%, wherein said percentages are with respect to the total weight of the compound.

Said (a.2) analogue of a vegetable chondroitin sulfate may comprise said extract of at least one alga (Ulva lactuca and/or Fucus) in a percentage by weight comprised from 30% to 95% with respect to the total weight of (a.2) (for example about 35%, 40%, 50%, 60%, 70% or 80% or 90%), preferably from 40% to 95%, more preferably from 60% to 90%; and/or wherein said extract of at least one alga comprises an extract of Ulva lactuca and an extract of Fucus (preferably Fucus vesiculosus) in a by weight ratio or by percentage p/p (e.g. powdered product) Ulva lactuca:Fucus=from about 2:1 to 1:2, preferably 1:1.

Said (a.2) analogue of a vegetable chondroitin sulfate may comprise said extract of at least one alga (Ulva lactuca and/or Fucus) in a percentage by weight comprised from 30% to 95% with respect to the total weight of (a.2) (for example about 40%, 50%, 60%, 70%, 80% or 90%; Ulva lactuca:Fucus percentage ratio=from about 2:1 to 1:2, preferably 1:1, if both present) and, optionally, said hyaluronic acid or a salt thereof in a percentage by weight comprised from 1% to 30% with respect to the total weight of (a.2) (for example about 2%, 3%, 4%, 5%, 6%, 7%, 8%, 9%, 10%, 15%, or 20%), and optionally at least one additive and/or excipient; for example said extract of at least one alga (Ulva lactuca and/or Fucus) from 60% to 90% and, optionally, hyaluronic acid or a salt thereof from 1% a 20%, or said extract of at least one alga (Ulva lactuca and/or Fucus) from 65% to 85% and hyaluronic acid or a salt thereof from 1% to 15%, or said extract of at least one alga (Ulva lactuca and/or Fucus) from 70% to 80% and, optionally, hyaluronic acid or a salt thereof from 1% to 10%.

Said mixture of the invention may comprise or, alternatively, consist of:
(a.1) a chondroitin sulfate of plant origin or a salt thereof, preferably a sodium chondroitin sulfate, having an average molecular weight comprised from 1 kDa to 250 kDa or 100 kDa, preferably from 1 kDa to less than or equal to 50 kDa, preferably from 1 kDa to 20 kDa (e.g. From 5 kDa to 15 kDa), or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a hyaluronic acid or an acceptable pharmaceutical or food grade salt thereof, for example a hyaluronic acid or a salt thereof having an average molecular weight from about 1 kDa to about 2000 kDa.

Besides (a.1) a vegetable chondroitin sulfate or (a.2) an analogue of a vegetable chondroitin sulfate (i.e. a mixture comprising an extract of Ulva lactuca and/or Fucus and, optionally, a hyaluronic acid or a salt thereof) and, optionally, (c) a basic substance, said (b) hyaluronic acid or a salt thereof, present in the mixture of the invention, is preferably a hyaluronic acid (linear or branched) having an average molecular weight comprised from about 1 kDa to about 10,000 kDa, preferably from about 1 kDa to about 2000 kDa, more preferably from about 200 kDa to about 1500 kDa (for example 300 kDa, 500 kDa, 550 kDa, 700 kDa, 800 kDa, 900 kDa, 1000 kDa, 1200 kDa, or 1400 kDa), even more preferably from about 400 kDa to about 1000 kDa, or from about 600 kDa to about 800 kDa; advantageously it is a sodium hyaluronate having an average molecular weight comprised from 400 kDa to 800 kDa (example, CAS No. 9067-32-7).

In the context of the present invention the expression “hyaluronic acid” is used to indicate both the hyaluronic acid as such and an acceptable pharmaceutical or food grade salt thereof. Said hyaluronic acid salt is preferably an alkali metal or an alkaline earth metal salt, for example selected from the group comprising or, alternatively, consisting of sodium hyaluronate, potassium hyaluronate, calcium hyaluronate and magnesium hyaluronate; more preferably sodium hyaluronate.

In an embodiment, the mixture of the invention may comprise or, alternatively, consist of:
(a.1) a chondroitin sulfate of plant origin or a salt thereof, preferably a sodium chondroitin sulfate, having an average molecular weight comprised from 1 kDa to ≤50 kDa (e.g. from 1 kDa to 20 kDa or from 5 kDa to 15 kDa), and
(b) a sodium hyaluronate having an average molecular weight from about 400 kDa to about 1000 kDa, or from about 600 kDa to about 800 kDa; advantageously it is a sodium hyaluronate having an average molecular weight comprised from 400 kDa to 800 kDa (example, CAS No. 9067-32-7).

In an embodiment, the mixture of the invention may comprise or, alternatively, consist of:
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and in addition
(b) a sodium hyaluronate having an average molecular weight from 200 kDa to 1500 kDa, preferably a sodium hyaluronate having an average molecular weight from about 400 kDa to about 1000 kDa, or from about 600 kDa to about 800 kDa; advantageously it is a sodium hyaluronate having an average molecular weight comprised from 400 kDa to 800 kDa (example, CAS No. 9067-32-7).

In particular, the mixture of the invention may comprise or, alternatively, consist of:
(a.2) an analogue of a vegetable chondroitin, such as a compound comprising a mixture comprising or, alternatively, consisting of an extract of Ulva lactuca and Fucus (preferably Fucus vesiculosus) and, optionally, a hyaluronic acid or a salt thereof (preferably sodium hyaluronate), and in addition
(b) a sodium hyaluronate having an average molecular weight from 200 kDa to 1500 kDa, preferably a sodium hyaluronate having an average molecular weight from about 400 kDa to about 1000 kDa, or from about 600 kDa to about 800 kDa; advantageously it is a sodium hyaluronate having an average molecular weight comprised from 400 kDa to 800 kDa (example, CAS No. 9067-32-7).

Said mixture of the invention may comprise or, alternatively, consist of:
(a.1) a chondroitin sulfate of plant origin or a salt thereof, preferably a sodium chondroitin sulfate, having an average molecular weight comprised from 1 kDa to 250 kDa, preferably from 1 kDa a≤50 kDa (e.g. from 1 kDa to 20 kDa or from 5 kDa to 15 kDa), or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a hyaluronic acid or a salt thereof, preferably sodium hyaluronate (having the described molecular weights), and
(c) a basic substance with antacid properties, such as a salt selected from the group comprising or, alternatively, consisting of: an oxide salt, a hydroxide salt, a carbonate salt, a bicarbonate salt, a silicate salt, a trisilicate salt, a sulfate salt, a citrate salt and mixtures thereof, wherein said salt is an alkali metal cation or an alkaline earth metal cation or a metal cation salt (3+ or III).

According to a preferred example, said mixture of the invention may comprise or, alternatively, consist of:
(a.1) a chondroitin sulfate of plant origin or a salt thereof, preferably a sodium chondroitin sulfate, having an average molecular weight comprised from 1 kDa to ≤50 kDa (e.g. from 1 kDa to 20 kDa or from 5 kDa to 15 kDa), or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a hyaluronic acid or a salt thereof, preferably sodium hyaluronate (having the described molecular weights), and
(c) a basic substance, wherein said basic substance is selected from the group comprising or, alternatively, consisting of aluminium hydroxide, magnesium hydroxide, magnesium trisilicate, sodium hydroxide and a mixture thereof.

In a preferred embodiment of the mixture or composition of the present invention, the components [(b) hyaluronic acid or salt thereof]:[(c) basic substance]:[(a.1) or (a.2) vegetable chondroitin or analogue thereof] (in short HA:basic substance:CS), such as for example [HA:Al(OH)₃:CS] or [HA:Mg trisilicate:CS] or [HA:Mg(OH)₂:CS], are in a [HA:basic substance:CS] weight ratio approximately comprised from 1:10:20 to 1:30:60, preferably from 1:15:30 to 1:25:50, more preferably in a [HA:basic substance:CS] weight ratio approximately 1:20:40.

In an embodiment, the mixture of the invention comprises or, alternatively, consists of:
(b) a hyaluronic acid, or a salt thereof, in a percentage by weight comprised from 0.1% to 5%; preferably from 0.5% and 2%, with respect to the total weight of the mixture;
(a.1) a vegetable chondroitin sulfate (or a salt thereof) having an average molecular weight comprised from 1 kDa to ≤50 kDa (e.g. from 1 kDa to 20 kDa or from 5 kDa to 15 kDa), or (a.2) an analogue of a vegetable chondroitin sulfate (mixture of an extract of Ulva lactuca and/or Fucus and, optionally, a hyaluronic acid), in a percentage by weight comprised from 20% to 80%, preferably from 30% to 70%, with respect to the total weight of the mixture and, optionally;
(c) an antacid substance in a percentage by weight comprised from 10% to 50%; preferably from 20% to 40%, with respect to the total weight of the mixture.

For example, the mixture of the invention may comprise or, alternatively, consist of:
(a.1) a chondroitin sulfate of plant origin or a salt thereof, preferably a sodium chondroitin sulfate, having an average molecular weight comprised from 1 kDa to ≤50 kDa (e.g. from 1 kDa to 20 kDa or from 5 kDa a 15 kDa); or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a hyaluronic acid or a salt thereof, preferably sodium hyaluronate, having an average molecular weight from 400 kDa to 600 kDa or from 600 kDa to 800 kDa, and
(c) an aluminium hydroxide; preferably a mixture of aluminium hydroxide and sodium bicarbonate, in a 2:1 by weight ratio.

In an embodiment, the mixture of the invention may comprise or, alternatively, consist of:
(a.1) a chondroitin sulfate of plant origin or a salt thereof, preferably a sodium chondroitin sulfate, having an average molecular weight comprised from 1 kDa to ≤50 kDa (e.g. from 1 kDa to 20 kDa or from 5 kDa to 15 kDa), or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a hyaluronic acid or a salt thereof, preferably sodium hyaluronate, having an average molecular weight from 400 kDa to 600 kDa or from 600 kDa to 800 kDa, and
(c) a magnesium hydroxide; preferably a mixture of aluminium hydroxide and sodium bicarbonate, in a 2:1 by weight ratio.

In an embodiment, the mixture of the invention may comprise or, alternatively, consist of:
(a.1) a chondroitin sulfate of plant origin or a salt thereof, preferably a sodium chondroitin sulfate, having an average molecular weight comprised from 1 kDa to ≤50 kDa (e.g. from 1 kDa to 20 kDa or from 5 kDa to 15 kDa), or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a hyaluronic acid or a salt thereof, preferably sodium hyaluronate, having an average molecular weight from 400 kDa to 600 kDa or from 600 kDa to 800 kDa, and
(c) a magnesium trisilicate, preferably a mixture of magnesium trisilicate and sodium bicarbonate in a 2:1 by weight ratio; for example, said magnesium trisilicate may be a magnesium trisilicate in hydrated form (example CAS No. 14987-04-3, formula: 2MgO·3SiO3·aq).

Alternatively, said mixture of the invention may comprise or, alternatively, consist of:
(a.1) a chondroitin sulfate of plant origin or a salt thereof, preferably a sodium chondroitin sulfate, having an average molecular weight comprised from 1 kDa to ≤50 kDa (e.g. from 1 kDa to 20 kDa or from 5 kDa to 15 kDa), or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(c) a basic substance, wherein said basic substance is selected from the group comprising or, alternatively, consisting of aluminium hydroxide, magnesium hydroxide, magnesium trisilicate, sodium hydroxide and a mixture thereof.

A second aspect of the present invention relates to a composition (in short, composition of the invention) comprising: said mixture of the invention and at least one acceptable pharmacological or food grade additive and/or excipient, wherein said mixture comprises or, alternatively, consists of said (a.1) a low molecular weight vegetable chondroitin sulfate or (a.2) an analogue of a vegetable chondroitin (i.e. an alga/algae extract and, optionally, a hyaluronic acid) and, optionally, (b) a hyaluronic acid and/or (c) a basic substance and/or further ingredients, according to any one of the described embodiments or aspects.

Said at least one pharmaceutical or food grade additive and/or excipient, comprised in the composition of the invention together with the mixture of the invention comprising (a.1) or (a.2) and optionally (b) and/or (c), consists of a substance devoid of therapeutic activity suitable for pharmaceutical or food use selected from ancillary substances known to the person skilled in the art such as, for example diluents, solvents (including water, glycerine, ethyl alcohol), solubilisers, thickeners, sweeteners, flavour enhancement agents, colorants, lubricants, surfactants, antimicrobials, antioxidants, preservatives, pH stabilising buffers and mixtures thereof. Said additives and/or excipients vary with the variation of the pharmaceutical or food form of the composition of the invention (for example, liquid or solid).

Said mixtures or compositions of the invention, comprising (a.1) or (a.2) and, optionally, (b) and/or (c) and/or further ingredients according to any aspect or embodiment described, may be pharmaceutical mixtures or compositions, medical device mixtures or compositions (Medical Device Regulation (EU) 2017/745 (MDR)), dietary supplements and/or foods for special medical purposes (FSMPs).

The mixtures or compositions of the invention, comprising (a.1) or (a.2) and, optionally, (b) and/or (c) and/or further ingredients according to any aspect or embodiment described, do not comprise bioadhesive polymers such as for example, poloxamers, and/or ethylene and propylene oxide copolymers (example of commercial product Lutrol®, such as Lutrol® F127), and/or vinylpyrrolidone or polyvinylpyrrolidone polymers.

A third aspect of the present invention relates to said mixtures or compositions of the invention, comprising said (a.1) or (a.2) and, optionally, (b) and/or (c) and further ingredients according to any one of the aspects or embodiments described, for use as medicament; preferably for use in a method for the preventive or curative treatment of diseases and/or symptoms of the oral mucous membrane and/or the upper respiratory tract, the mucous membrane of the laryngopharyngeal tract and the mucous membrane of the gastro-oesophageal tract, such as for example diseases and/or symptoms caused by gastric reflux in the gastroesophageal tract or in extra-oesophageal regions, and ulcers, lacerations and/or inflammations caused in the mucous membrane or in the lining tissues of the various anatomical regions present in the tract from the stomach to the oral cavity, also including the upper respiratory tract. Said treatment method provides for the administration of a mixture or composition of the present invention in a therapeutically effective amount to a subject in need.

Said diseases and/or symptoms may be selected from the group comprising or, alternatively, consisting of:
gastroesophageal reflux disease (GERD), laryngopharyngeal reflux disease (LPR), lesions or ulcers in the mucosa or in the tissues of the oral cavity (tongue and/or palate included), lesions or ulcers in the mucosa or in the tissues of the pharyngolaryngeal tract, lesions or ulcers in the mucosa or in the tissues of the oesophageal tract, oesophageal ulcers, deepithelialization of the oesophageal mucosa, peptic ulcers, and/or gastric ulcers; and/or
mucositis, aphthous and/or aphthoid lesions; and/or
acute or chronic inflammations of the mucous membrane or tissues of the oral cavity, acute or chronic inflammations of the mucous membrane/of the tissues of the pharyngolaryngeal tract, acute or chronic inflammations of the mucous membrane or tissues of the oesophageal tract and/or oesophagitis (acute or chronic inflammation of the oesophageal mucosa); and/or
acid regurgitation, heartburn, feeling of gastric fullness, epigastric pain, dyspepsia, nausea, chronic cough, bronchospasm, inflamed throat, laryngitis, globus sensation or hypopharyngeal bolus, pyrosis, dysphonia and/or nasopharyngeal inflammation, laryngospasm, raucousness, globus pharyngis, dysphonia, dysphagia, excessive throat clearing, sore or burning throat, postnasal drip, pharyngitis and/or other symptoms of the upper respiratory tract.

The characteristic symptoms of gastric reflux can be divided into typical (e.g. acid regurgitation, heartburn), atypical (e.g. feeling of gastric fullness, epigastric pain, dyspepsia, nausea) and extra-oesophageal (e.g. chronic cough, bronchospasm, laryngospasm, raucousness, globus pharyngis, dysphonia, dysphagia, excessive throat clearing, sore or burning throat, inflamed throat, postnasal drip, laryngitis, pharyngitis), and/or other symptoms of the upper respiratory tract. Said typical, atypical and/or extra-oesophageal gastric reflux symptoms may be treated using mixtures or compositions of the present invention.

The mixtures or compositions of the invention may be for use in a method for the preventive or curative treatment of diseases and/or symptoms of the mucous membranes or in the lining tissues of the various anatomical regions present in the tract from the stomach to the oral cavity (as defined above, also comprising the upper respiratory tract) both when administered to a subject as a single therapy, and when administered as an adjuvant of at least another therapy or composition capable of treating said disorders, such as for example a H2 antihistamine, a prokinetic agent, an antacid, an alginate and/or a proton pump inhibitor (in short, PPI) for the treatment of gastric reflux, wherein said PPI is preferably selected from the group of PPIs comprising or, alternatively, consisting of: omeprazole, lansoprazole, esomeprazole, pantoprazole, rabeprazole sodium, ilaprazole and tenatoprazole.

According to an embodiment, besides (a.1) or (a.2) and, optionally, (b) and/or (c) and further ingredients, the composition of the invention may further comprise a proton pump inhibitor (PPI) selected from the aforementioned group of PPIs.

Advantageously, the mixtures or the compositions of the invention are formulated for oral administration. The dosage form of the mixtures or compositions of the invention may be a solid form, such as tablet, chewable tablet, tablet to be dissolved in the mouth without chewing (or to be sucked or melt-in mouth tablet or mouth dissolvable), capsule, granules or powder (for example, granules or powder to be dissolved in a water-based liquid, or mouth dissolvable granules or powder), dry powder for oral or nasal inhalation, or a semi-solid form, such as soft-gel, or a liquid form, such as solution, suspension, dispersion, emulsion or syrup.

In a first embodiment, said mixtures or compositions of the invention for oral administration, comprising (a.1) or (a.2) and, optionally, (b) and/or (c) and/or further ingredients according to any aspect or embodiment described, are formulated in solid form of tablets, such as tablet as such, chewing tablet or tablet to be dissolved in the mouth, preferably tablet to be chewed or to be dissolved in the mouth.

The compositions of the invention in solid form of tablets according to said first embodiment, preferably of tablets to be chewed or to be dissolved in the mouth, may comprise (for example according to Table 1 or Table 2) a mixture comprising or, alternatively, consisting of:
(a.1) a chondroitin sulfate of plant origin or a salt thereof, preferably a sodium chondroitin sulfate, having an average molecular weight comprised from 1 kDa to 250 kDa, preferably from 1 kDa a≤50 kDa (e.g. from 1 kDa to 20 kDa or from 5 kDa to 15 kDa), or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a hyaluronic acid or a salt thereof, preferably sodium hyaluronate, having an average molecular weight from 400 kDa to 600 kDa (or from 600 kDa to 800 kDa), and
(c) a basic substance, wherein said basic substance is selected from the group comprising or, alternatively, consisting of aluminium hydroxide, magnesium hydroxide, magnesium trisilicate, and a mixture thereof;
furthermore, the composition comprising at least one additive and/or excipient suitable for the form of a tablet or tablet to be chewed or tablet to be sucked in the mouth.

The compositions of the invention in solid form of tablets, preferably of tablet to be chewed or to be dissolved in the mouth, preferably comprise a mixture comprising or, alternatively, consisting of:
(a.1) a chondroitin sulfate of plant origin or a salt thereof, preferably a sodium chondroitin sulfate, having an average molecular weight comprised from 1 kDa to ≤50 kDa (e.g. from 1 kDa to 20 kDa or from 5 kDa to 15 kDa), or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a sodium hyaluronate, having an average molecular weight from 400 kDa to 600 kDa (or from 600 kDa to 800 kDa), and
(c) magnesium hydroxide;
furthermore, the composition comprising at least one additive and/or excipient suitable for the form of a tablet or tablet to be chewed or tablet to be sucked in the mouth.

The compositions of the invention in solid form of tablets, preferably of tablet to be chewed or to be dissolved in the mouth, preferably comprise a mixture comprising or, alternatively, consisting of:
(a.1) a chondroitin sulfate of plant origin or a salt thereof, preferably a sodium chondroitin sulfate, having an average molecular weight comprised from 1 kDa to ≤50 kDa (e.g. from 1 kDa to 20 kDa or from 5 kDa to 15 kDa), or
(a.2) an analogue of a vegetable chondroitin, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a sodium hyaluronate, having an average molecular weight from 400 kDa to 600 kDa (or from 600 kDa to 800 kDa), and
(c) magnesium trisilicate, for example magnesium trisilicate in hydrated form (e.g. CAS No. 14987-04-3); furthermore, the composition comprising at least one additive and/or excipient suitable for the form of a tablet or tablet to be chewed or tablet to be sucked in the mouth.

Preferably, in said first embodiment, the weight ratio (by weight) between the components of the mixture of the invention [HA:basic substance:CS], such as for example [HA:Al(OH)₃:CS] or [HA:Mg trisilicate:CS] or [HA:Mg(OH)₂:CS], is comprised from 1:10:20 to 1:30:60, preferably from 1:15:30 to 1:25:50, more preferably about 1:20:40.

According to the first embodiment of the composition of the present invention, each unit in the form of a tablet as such, to be chewed or to be dissolved in the mouth comprises hyaluronic acid or a salt thereof, preferably sodium hyaluronate, in an amount comprised from 5 mg to 100 mg, preferably from 5 mg to 50 mg; the vegetable chondroitin sulfate (a.1) or an analogue thereof (a.2) in an amount comprised from 200 mg to 700 mg, preferably from 200 mg to 450 mg; and aluminium hydroxide in an amount comprised from 150 mg to 325 mg, advantageously from 150 mg to 250 mg. For example, each unit of the composition of the invention in the form of a tablet as such, to be chewed or to be dissolved in the mouth comprises hyaluronic acid or a salt thereof, preferably sodium hyaluronate, in an amount comprised from 5 mg to 25 mg, vegetable chondroitin sulfate (a.1) or an analogue thereof (a.2) in an amount comprised from 300 mg to 400 mg, and aluminium hydroxide in an amount comprised from 150 mg to 200 mg.

The compositions of the invention in the form of a tablet as such, to be chewed or dissolved in the mouth advantageously comprise from 0.5% to 1.5% of hyaluronic acid (b), from 30% to 40% of vegetable chondroitin sulfate (a.1) or an analogue thereof (a.2), and from 15% to 20% of aluminium hydroxide or magnesium trisilicate (c).

The compositions of the invention according to said first embodiment (i.e. tablet form) may comprise an alkaline-earth metal carbonate, such as for example a calcium carbonate or a magnesium carbonate. Furthermore, the compositions of the invention according to said first embodiment may further comprise a glycyrrhizic acid or a salt thereof, such as for example the ammonium or potassium salt thereof.

In said first embodiment of the composition of the invention in the form of a tablet as such, to be chewed, or to be dissolved in the mouth, said at least one additive and/or excipient is selected from the group comprising or, alternatively, consisting of: starches, for example cellulose and derivatives thereof; lubricants, for example talc, stearic acid and magnesium stearate; diluents, for example talc, powdered cellulose, lactose, polyethylene glycol, starches (e.g. corn or wheat starch), mannitol; disaggregating agents, for example microcrystalline cellulose or crospovidone; binders, for example methylcellulose, sodium carboxymethylcellulose; sweeteners or flavour enhancement agents, for example natural or synthetic oils.

The tablets as such, to be chewed or to be dissolved in the mouth, may further comprise ingredients for controlled release such as for example glyceryl behenate (e.g. products marketed under the names Compritol® 888 ATO and Compritol® E ATO), stearoyl macroglycerides (e.g. product marketed under the name Gelucire®), or glyceryl palmitostearate (e.g. product marketed under the name Precirol® ATO 5).

The sweeteners comprised in the tablets to be chewed or to be dissolved in the mouth according to said first embodiment may be natural sugars, optionally reduced, such as for example sucrose, dextrose, xylitol, mannitol or sorbitol, or a synthetic product such as sodium saccharin or aspartame.

The synthetic sweeteners may be present in a percentage by weight comprised from 0.1% to 5%, while the natural sugar, optionally reduced, may be present in a percentage by weight comprised from 10% to 20%, preferably from 15% to 20%, wherein said percentages are with respect to the total weight of the composition of the invention.

Flavour enhancement agents, generally supported on a solid matrix, may be present in a percentage by weight comprised from 0.5% to 1.5% with respect to the total weight of the composition of the invention.

According to one aspect of said first embodiment, the chewable tablets are broken into the mouth by chewing and dispersed in the saliva so as to be swallowed. Alternatively, according to a further aspect of said first embodiment, the chewable tablets have the consistency of a rubber.

The advantage of formulating the mixture or compositions of the invention in the form of a tablet to be dissolved in the mouth without chewing it (solid monolithic tablet), and without swallowing it whole or in pieces, is due to the fact that saliva starts to dissolve the vegetable chondroitin sulfate (a.1) or said analogue thereof (a.2), and optionally hyaluronic acid (b), by embedding a part of the same in the saliva and therefore forming a gelatinous substance comprising the active ingredients of the composition of the invention having viscosity and adhesiveness properties. Once swallowed, said gelatinous substance (or viscous gel) is able to progressively, uniformly and continuously line the oral cavity, the laryngo-pharynx and the oesophagus until it reaches the stomach gradually. The lining of the mucous membranes with said gelatinous substance carries out an action of protecting the mucous membranes, has a healing effect of the mucous membranes, and has a buffer effect on the acidity present on the mucosa.

Furthermore, with the dissolution of the composition in solid form, the production of further saliva is triggered due to the stimulation of the salivary glands.

For this reason, it is preferable that the composition of the invention in the form of a tablet to be dissolved in the mouth be not swallowed as such, nor crushed into smaller pieces, nor swallowed in small pieces. For example, it is preferable that said tablet to be dissolved in the mouth remains in the mouth so as to be dissolved slowly over a period of time comprised from 1 minute to 60 minutes, preferably from 3 minutes to 40 minutes, more preferably from 5 minutes to 20 minutes.

In a second embodiment, said compositions of the invention for oral administration, comprising (a.1) or (a.2) and, optionally, (b) and/or (c) and/or further ingredients according to any aspect or embodiment described, are formulated in water-based or hydroalcoholic liquid form, preferably in form of syrup.

The compositions of the invention in water-based or hydroalcoholic liquid form may comprise (for example according to Table 3) a mixture comprising or, alternatively, consisting of:
(a.1) a vegetable chondroitin sulfate or a salt thereof, preferably a sodium chondroitin sulfate, having an average molecular weight comprised from 1 kDa to 250 kDa, preferably from 1 kDa a≤50 kDa (e.g. from 1 kDa to 20 kDa or from 5 kDa to 15 kDa), or
(a.2) an analogue of a vegetable chondroitin sulfate, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a hyaluronic acid or a salt thereof, preferably sodium hyaluronate, having an average molecular weight from 400 kDa to 600 kDa (or from 600 kDa to 800 kDa), and optionally
(c) a basic substance, for example sodium hydroxide;
furthermore, the composition comprises at least one additive and/or excipient suitable for a water-based or hydroalcoholic liquid form, preferably a syrup form.

Preferably, in said second embodiment of a water-based or hydroalcoholic liquid, the weight ratio (by weight) between the components of the mixture or composition of the invention, hyaluronic acid (b) toward vegetable chondroitin sulfate (a.1) or an analogue thereof (a.2) (or salts thereof), in short [HA:CS], is comprised from 1:3 to 1:20, preferably from 1:5 to 1:15, more preferably about 1:10. Should said composition comprise a basic substance (c), for example sodium hydroxide, the [basic substance:HA:CS] weight ratio, such as for example [NaOH:HA:CS], is comprised from 0.01:1:3 to 0.5:1:20, preferably from 0.05:1:5 to 0.5:1:15, more preferably about 0.1:1:10.

The compositions of the invention in water-based or hydroalcoholic liquid form may comprise (for example according to Table 3) a mixture comprising or, alternatively, consisting of:
(a.1) a vegetable chondroitin sulfate or a salt thereof, preferably a sodium chondroitin sulfate, having an average molecular weight comprised from 1 kDa to 250 kDa, preferably from 1 kDa a≤50 kDa (e.g. from 1 kDa to 20 kDa or from 5 kDa to 15 kDa), or
(a.2) an analogue of a vegetable chondroitin sulfate, such as a compound comprising a mixture comprising or, alternatively, consisting of an extract of Ulva lactuca and/or Fucus (preferably Fucus vesiculosus) and, optionally, a hyaluronic acid or a salt thereof (preferably sodium hyaluronate), and furthermore in addition to (a.1) or (a.2)
(b) a hyaluronic acid or a salt thereof, preferably sodium hyaluronate, having an average molecular weight from 400 kDa to 600 kDa (or from 600 kDa to 800 kDa), and optionally
(c) a basic substance, for example sodium hydroxide;
furthermore, the composition comprises
(d) honey, and
(e) an Aloe vera gel,
and at least one additive and/or excipient suitable for a water-based or hydroalcoholic liquid form, preferably a syrup form.

In a first aspect of said second embodiment, the composition is a water-based solution.

In a second aspect of said second embodiment, the composition is a hydroalcoholic liquid solution. Advantageously said hydroalcoholic liquid composition comprises at least one alcohol in a percentage by volume comprised from 0.01% to 3%, preferably comprised from 0.05% to 2.5%, more preferably from 0.1% to 1.0%, wherein said percentages are with respect to the total volume of the composition.

Advantageously, said at least one alcohol is an ethyl alcohol or any other alcohol suitable for forming a hydroalcoholic solution which can be administered to human or animal subjects and it is also compatible with the components from (a) to (e) present in the composition.

For example, the composition of the present invention in form of syrup may have a specific weight of about 1.2-1.3 kg/dm³ at 20° C. and a viscosity of about 200-205 mPa·s at 20° C.

In the context of the present invention, the expression “honey” is used to indicate the natural sweet product made by bees (e.g. Apis mellifera), starting from nectar of one or more plant varieties of any kind or from secretions coming from live parts of plants or from substances secreted by sucking insects found on live parts of plants, which bees collect, transform, combine them with their own specific substances, deposit, dehydrate, store and allow to mature in the honeycombs, according to the definition of Italian Legislative Decree n° 179 dated 21 May 2004 implementing directive 2001/110/EC on the production and marketing of honey. Said honey can be obtained by means of standard processes and equipment known to the person skilled in the art (for example comprising extraction, extraction of honey, decantation, filtration, guided crystallisation and similar operations). In the context of the present invention, the expression “honey” also includes products that can be obtained from natural honey, including those for industrial use, for example by refining processes or thermal treatments, such as pasteurisation.

According to an aspect of said second embodiment, the expression “honey” is used to indicate the above, provided that said honey is not honey with non-peroxidic antibacterial activity and/or provided that said honey is unfiltered clover honey and processed at a temperature from 100° F. to 140° F.

By way of non-limiting example, the honey that can be used in the composition of the present invention can have a pH from 3.5 to 4.5, a weight loss on drying of 18% (by weight with respect to the total weight of the honey) and a reducing sugar content on the dry matter of 70% (by weight based on the total weight of honey).

In the context of the present invention, the expression “Aloe vera gel” is used to indicate the mucilaginous gel obtained from the parenchymal tissue of Aloe vera leaves (L) Burm. f. or Aloe barbadensis (Mill). In the Aloe vera gel monograph in “WHO monographs on selected medicinal plants” (Vol. 1 World Health Organization, Geneva, 1999 pages 43-49) it is reported that the major components thereof, besides water, are polysaccharides (pectins, emi-celluloses, glucomannans, acemannans and mannose derivatives), and that the administration of Aloe vera gel would not have shown any relevant therapeutic effect. Furthermore, in the context of the present invention, the expression “Aloe vera gel” should not be confused with the general expression “Aloe vera”, given that Aloe vera gel is a specific part obtained from the Aloe vera plant, such as mucilaginous gel obtained from the parenchymal tissue of Aloe vera leaves (L) Burm. f. or Aloe barbadensis Miller. Aloe vera gel has a different composition and properties from Aloe vera juice, given that the juice is obtained from the same plant but with different methods, such as cutting and drying.

By way of non-limiting example, the Aloe vera gel used in the present invention may have a pH from 3 to 6 (for example 3.5, 4, 4.5, 5 or 5.5), preferably from 3.7 to 4.2, and it may have an aloin content less than 1 ppm. The Aloe vera gel of the present invention is obtained using standard preparation methods. An example of a preparation method is as follows: Aloe gel is obtained using the following steps: selection of leaves of adult Aloe plants; removal of the outer part, where the thorns and the related glands secreting the anthraquinones are concentrated (these substances are particularly known for their laxative action); squeezing of the Aloe pulp, from which the gel is obtained to the detriment of the fibrous residues; stabilisation of the gel with special additives to protect the active ingredients from chemical oxidation; purification of the gel; packaging in airtight, dark or opaque containers, given that Aloe gel degrades easily if exposed to oxygen and direct light; storage in a cool place (4-20° C.), given that Aloe gel is strongly compromised by high temperatures. Alternatively, Aloe vera gel may be prepared starting from inside of Aloe vera leaves, freeze-dried according to the methods and equipment known to the person skilled in the art.

According to one aspect of said second embodiment, the liquid composition of the present invention (e.g. water or hydroalcoholic-based syrup) may comprise:
said (a.1) low molecular weight vegetable chondroitin sulfate (CS) or a salt thereof (e.g. Na-CS) or said (a.2) an analogue of vegetable chondroitin sulfate (i.e. a mixture comprising an extract of Ulva lactuca and/or Fucus and, optionally, HA or Na-HA), wherein said (a.1) or said (a.2) are in a percentage comprised from about 0.1% to about 10%, preferably comprised from about 1% to about 4%;
(b) a hyaluronic acid (HA) or a salt thereof, preferably Na-HA, in a percentage comprised from about 0.01% to about 5%, preferably comprised from about 0.1% to about 0.5%;
(d) a honey in a percentage comprised from 5% to 50%, preferably comprised from about 10% to about 40%; and
(e) an Aloe vera gel in a percentage comprised from about 0.01% to about 5%, preferably comprised from about 0.1% to about 0.5%;
wherein said percentages are percentages by weight with respect to the total weight of the composition.

According to a preferred aspect of said second embodiment, the liquid composition of the present invention (e.g. water or hydroalcoholic-based syrup) may comprise:
said (a.1) low molecular weight vegetable chondroitin sulfate (CS) or a salt thereof (e.g. Na-CS) or said (a.2) an analogue of vegetable chondroitin sulfate (i.e. a mixture comprising an extract of Ulva lactuca and/or Fucus and, optionally, HA or Na-HA), in an amount comprised from 1 g to 5 g, preferably comprised from 2 g to 3 g;
(b) a hyaluronic acid (HA) or a salt thereof, preferably HA sodium salt (Na-HA), in an amount comprised from 100 mg to 500 mg, preferably comprised from 100 mg to 300 mg;
(d) a honey in an amount comprised from 10 g to 50 g, preferably comprised from 20 g to 30 g; and
(e) an Aloe vera gel in an amount comprised from 100 mg to 500 mg, preferably comprised from 150 mg to 300 mg.

In an example of said second embodiment, the liquid composition of the present invention (e.g. water or hydroalcoholic-based syrup) comprises, per 100 ml of composition:
said (a.1) low molecular weight vegetable chondroitin sulfate (CS) or a salt thereof (e.g. Na-CS) or said (a.2) an analogue of vegetable chondroitin sulfate (i.e. A mixture comprising an extract of Ulva lactuca and/or Fucus and, optionally, HA or Na-HA): about 2.5 g;
(b) a hyaluronic acid or a salt thereof (e.g. Na-HA): about 0.2 g;
(d) a honey: about 25 g;
(e) a freeze-dried Aloe vera gel: about 0.25 g; and
additives and/or excipients (and optionally sodium hydroxide or analogue).

Examples of said additives and/or excipients potentially present in the compositions according to said second embodiment in liquid form are the ancillary substances acceptable for pharmaceutical or food use known to the person skilled in the art and suitable for the preparation of liquid forms for oral administration such as for example, diluents, solvents (including water, glycerine, ethyl alcohol), solubilisers, thickeners, sweeteners, flavour enhancement agents, colorants, lubricants, surfactants, antimicrobials, antioxidants, preservatives, pH stabilising buffers and mixtures thereof. Non-limiting examples of said additives and/or excipients are maltodextrins, phosphate buffers, basic substances such as sodium hydroxide, xanthan gum, guar gum, fructose, natural or artificial flavours.

The compositions in liquid form of the present invention (e.g. water or hydroalcoholic-based syrup) allow a better contact of the individual components/substances with the oro-pharyngo-laryngo-oesophageal wall, facilitating their protection, lubrication and repair of the relative mucous membranes and/or tissues:
chondroitin sulfate, by adhering to the oro-pharyngo-laryngo and/or gastro-oesophageal mucosa, protects it by effectively isolating it from gastric juices or from the vapours thereof or from other inflammatory or damaging agents;
hyaluronic acid, combined with the adhesive properties of chondroitin sulfate, protects damaged gastric tissue and facilitates the reintegration thereof;
honey exerts a soothing, moisturising and protective action due to the formation of a film which covers the oesophageal and/or oro-pharyngo-laryngeal mucosa, acting as a barrier, protecting the irritated mucosa and moisturising it, thus alleviating the sensations of pain;
Aloe vera gel exerts a refreshing, soothing and protective action on the oesophageal and/or oro-pharyngo-laryngeal mucosa, exerting a barrier effect thanks to the mucopolysaccharides contained in it which form a sort of film on the mucous membranes.

The mixtures and compositions of the invention, according to any one of the aspects or embodiments described (e.g. in solid form of tablets, inhalation powder or liquid), have the following properties:
Moisturising, humectant, lubricant, viscosifier, film-forming, protective, with a barrier effect, due to the formation of a viscous layer, which covers the oesophageal and/or oro-pharyngo-laryngeal mucosa (and upper respiratory tract) by gripping said mucous membranes (“gripping” action) and thereby exerting a barrier action, useful to prevent the mucous membranes from coming into contact with external agents, and to help alleviate the irritation conditions thereof and thus to facilitating proper trophism and correct functions thereof. By virtue of these effects, the aforementioned invention is capable of exerting a trophic, anti-inflammatory, antalgic (anti-pain), repair (cicatrization), restoration, re-epithelization (promotes tissue regeneration) action;
Emollient and soothing preferentially on the epithelium and on the oro-pharyngo-laryngeal mucosa (and upper respiratory tract) and oesophageal mucosa, useful to relieve the most common symptoms of GERD and/or laryngopharyngeal reflux and to calm cough and other related symptoms. By virtue of these effects, the aforementioned invention is capable of exerting a refreshing, balsamic, calming, sedative action on cough;
Protection of the damaged gastroesophageal tract and/or oro-laryngopharyngeal (and upper respiratory tract) and facilitating the regeneration of the damaged mucous membranes or tissue tongue) comprised), suitable to prevent damage induced by the irritating action due to contact with acidic substances contained in gastroesophageal regurgitation. By virtue of these effects, the aforementioned invention is capable of exerting a gastro-protection action.

For the sake of clarity, in order to achieve the object of the present invention, the active components of the mixture or composition of the present invention (such as (a.1) or (a.1), (b), (c), (d) and/or (e)) may also be administered separately or in groups (preferably in a time interval of 30 minutes-2-3 hours) and in any order.

In the context of the present invention, the expression “subject/s” are used to indicate mammals (animals and humans), preferably human subjects, such as men, women, elderly subjects, individuals engaged in sports activities at amateur, competitive or professional level and/or paediatric subjects.

The expression “therapeutically effective amount” is used to indicate the amount of mixture or compound or formulation which elicits the biological or medicinal response in a tissue, system or subject which is sought and defined by a person skilled in the art.

Unless otherwise specified, the expression mixture or composition comprises a component in an amount “comprised in a range from x to y” is used to indicate that said component can be present in the composition in all the amounts present in said range, even though not specified, extremes of the range comprised.

### EXAMPLES

1. Examples of compositions of the invention in solid form of tablets (as such) or tablets to be chewed or tablets to be dissolved in the mouth (melt-in-mouth tablets), according to Table 1 and Table 2 are reported below.

TABLE 1

| | mg/tab | mg/tab | mg/tab | mg/tab |
|---|---|---|---|---|
| Ingredients | C1 | C2 | C3 | C4 |
| vegetable sodium | 400 | 400 | 400 | 400 |
| chondroitin sulfate (a.1) | | | | |
| or analogue thereof (a.2) | | | | |
| Sodium hyaluronate (b) | 10 | 10 | 15 | 15 |
| Basic substance (c) | Al(OH)₃ | Mg- | Mg(OH)₂ | — |
| | 200 | trisilicate | 200 | |
| | | 200 | | |
| Calcium Carbonate | 100 | 100 | 100 | 100 |
| Liquorice flavour | 70 | 70 | 70 | 70 |
| Glyceryl-Dibehenate | 20 | 20 | 20 | 20 |
| (E471) | | | | |
| Silicon dioxide | 15 | 15 | 15 | 15 |
| Sucrose | 255 | 255 | 255 | 455 |
| Ammonium | 10 | 10 | 10 | 10 |
| Glycyrrhizinate | | | | |

| | |
|---|---|
| Total weight | 1100 mg |

TABLE 2
Quali-quantitative composition

| | mg/tab | mg/tab | mg/tab |
|---|---|---|---|
| Ingredients | C5 | C6 | C7 |
| vegetable sodium chondroitin sulfate | 400 | 500 | 600 |
| (a.1) or analogue thereof (a.2) | | | |
| Sodium hyaluronate (b) | 10 | 15 | 10 |
| Basic substance (c) | Al(OH)₃ | Al(OH)₃ | Al(OH)₃ |
| | 200 | 150 | 250 |
| Calcium Carbonate | 100 | 300 | 200 |
| Glyceryl-Dibehenate (E471) | 15 | 15 | 15 |
| Silicon dioxide | 10 | 15 | 15 |
| Sucrose | 360 | 450 | 450 |
| Ammonium Glycyrrhizinate | 5 | 5 | 5 |

2. Examples of compositions of the invention in the liquid form of syrup (Examples A, B and C) are reported below.

### Example A

A composition (C) according to the invention in form of syrup (total volume of 100 ml) comprising the following ingredients was prepared:
vegetable sodium chondroitin sulfate (a.1 or analogue thereof (a.2):2.8 g
Sodium hydroxide (c): 0.23 g
Honey (d): 25 g
Freeze-dried Aloe vera gel (e): 0.25 g
Fructose: 23.8 g
Hydroxypropyl methylcellulose: 3 g
Benzoates (preservatives): 0.15 g
Flavours: 0.3 g
Thickener: 0.1 g
Water: q. s. at 100 ml

### Example B

A composition (C) according to the invention in form of syrup (total weight of 100 g) comprising the following ingredients was prepared:
vegetable sodium chondroitin sulfate (a.1) or analogue thereof (a.2): 2.3 g
Sodium hydroxide (c): 0.24 g
Sodium hydroxide (c): 0.15 g
Honey (d): 21 g
Freeze-dried Aloe vera gel (e): 0.21 g
Fructose: 6.2 g
Potassium/dipotassium phosphate buffer: 2.1 g
Benzoates (preservatives): 0.15 g
Xanthan gum: 0.25 g
Flavours: 0.3 g
Maltodextrins: 11.6 g
Thickener (guar meal): 0.2 g
Water: 55 g
Other excipients: q.s. at 100 g

### Example C

Table 3 reports an example of a composition according to the invention in form of syrup having a pH value comprised from 6.5 to 7.5 and a density value comprised from 1.19 to 1.21 g/ml (measured at 25° C. with a pycometer in the collection container).

TABLE 3

| INGREDIENTS | % | Amt per dose | |
|---|---|---|---|
| Purified water | 55.307 | 6636.855 | mg |
| Honey | 21.090 | 2530.800 | mg |
| Maltodextrin | 11.577 | 1389.240 | mg |
| Fructose | 6.221 | 746.520 | mg |
| vegetable sodium chondroitin | 2.315 | 277.778 | mg |
| sulfate (a.1) or analogue | | | |
| thereof (a.2) | | | |
| Dipotassium phosphate | 1.400 | 168.000 | mg |
| Potassium phosphate | 0.700 | 84.000 | mg |
| Xanthan gum | 0.250 | 30.000 | mg |
| Sodium hyaluronate | 0.231 | 27.778 | mg |
| Freeze-dried aloe vera gel | 0.208 | 25.000 | mg |
| (Aloe barbadensis miller) | | | |
| Cream caramel flavour | 0.200 | 24.000 | mg |
| Guar seed meal (Cyamopsis | 0.200 | 24.000 | mg |
| tetragonoloba L. Taub.) | | | |
| Sodium hydroxide | 0.150 | 18.030 | mg |
| Sodium methylparaben | 0.120 | 14.400 | mg |
| Propyl 4-hydroxybenzoate | 0.030 | 3.600 | mg |
| sodium salt | | | |
| Total | 100.0000% | 12000.0000 | mg |

### Experimental Part

### “Film Forming” Ability on 3D Reconstructed Oesophageal Epithelium

### 1. Summary

The present study was conducted in order to evaluate the “film-forming” ability in an acid environment (pH 3.3) on the 3D reconstructed human oesophageal epithelium (HO2E/S/5), of several ingredients, alone and combined, and commercial products.

The logic for the “film forming” protocol was based on a quantitative approach published by Casiraghi et al. ((2017) “In vitro method to evaluate the barrier properties of medical devices for cutaneous use.” Toxicol Pharmacol. 90:42-50.) which uses caffeine penetration kinetics as a barrier permeability probe.

In this study, caffeine dosage was associated, at the end of the study, with the following reading:
Modification of the permeability of the tissues by carrying out the Lucifer Yellow test;
pH measurement on the epithelial surface.

The solutions of the tested compositions were applied locally on the surface of the epithelium for 15 minutes (realistic contact time with the mucous membrane in vivo) at room temperature. The ability of the products to prevent caffeine penetration was monitored after 15 minutes, 1 hour and 2 hours.

The “film-forming” ability was quantified as the percentage of reduction in caffeine passage compared to the negative control (saline).

### 2. Material

2.1. The reconstructed human esophageal epithelium (HO2E/S/5) of 0.5 cm 2 was used.
2.2. The following ingredients and products were tested:
negative control: saline solution
positive control: white petroleum jelly
Compound II (in short, C-II)
Compound II (in short, C-II)
Compound III (in short, C-III)
Sodium hyaluronate (in short, SH)
Aluminum hydroxide (in short, AH)
Magnesium Trisilicate (in short, MT)
Animal chondroitin sulfate (in short, animal CS)
Vegetable chondroitin sulfate II (in short, vegetable CS II)
Vegetable chondroitin sulfate II (in short, vegetable CS II)

### Composition of Tested Products:

Compound II (C-II), out of 1100 mg comprises: chicken sodium chondroitin sulfate 400 mg, sodium hyaluronate 10 mg, Mg-trisilicate 200 mg, calcium carbonate 100 mg, additives and/or excipients q.s.
Compound III (C-III), out of 1200 mg comprises: chicken sodium chondroitin sulfate 278 mg, sodium hyaluronate 28 mg, NaOH 18 mg, honey 2531 mg, Aloe vera gel 25 mg, additives and/or excipients q.s.
Compound III (C-III), out of 1200 mg comprises: chicken sodium chondroitin sulfate 278 mg, sodium hyaluronate 28 mg, NaOH 18 mg, honey 2531 mg, Aloe vera gel 25 mg, additives and/or excipients q.s.
vegetable CS I: vegetable sodium chondroitin sulfate having an average molecular weight of about 9 kDa obtained from plant source.
vegetable CS II: Extract of Ulva lactuca and Fucus vesiculosus, sodium hyaluronate and wheat maltodextrin.

### 3. Design of Experiment

On each tissue, previously moistened with 15 μL of saline solution, 30 μL of the test solutions and the negative control (saline solution) were applied directly and uniformly to the epithelial tissues for 15 minutes at room temperature. 50 μL of white petroleum jelly were applied to epithelial tissues, without moistening with a saline solution, for 15 minutes at room temperature. The protocol was carried out on tissues in triplicate.

After 15 minutes of incubation, without a washing step, 100 μl of 0.5% caffeine acid solution (1 mg of caffeine/cm²; pH 3.3) in bi-distilled water were applied on the treated epithelium for a total period of 2 hours at room temperature.

To quantify the permeability to caffeine, the receptor fluid (1 ml of saline solution) from the basolateral compartment was collected after 15 minutes, after 1 hour and after 2 hours (end of exposure) and the caffeine content was analysed using the UPLC-UV method.

After the 2-hour treatment with caffeine solution, pH on the apical surface was measured using litmus paper. Then the epithelial surface was washed with saline solution to remove the excess of the test substance subject of examination and the caffeine solution, and the permeability of the barrier was evaluated using the Lucifer Yellow paracellular passage.

In order to have the same relative ratio between the ingredients present in the finished products, the following amounts were weighed and dissolved in 3 mL of saline solution:

TABLE A

| | | | | Animan | Vegetable | Vegetable | Final |
|---|---|---|---|---|---|---|---|
| TEST | SH | AH | MT | CS | CS II | CS II | volume |
| P1 | 1100 mg of Compound I | | | | | | 3 ml |
| P2 | 10 mg | 200 mg | — | 400 mg | — | — | 3 ml |
| P3 | 10 mg | 200 mg | — | — | 400 mg | — | 3 ml |
| P4 | 10 mg | 200 mg | — | — | — | 400 mg | 3 ml |
| P5 | 1100 mg of Compound II. | | | | | | 3 ml |
| P6 | 10 mg | — | 200 mg | — | 440 mg | — | 3 ml |
| P7 | 10 mg | — | 200 mg | — | — | 440 mg | 3 ml |
| P8 | 10 mg | — | — | 440 mg | — | — | 3 ml |
| P9 | 10 mg | — | — | — | 440 mg | — | 3 ml |
| P10 | 10 mg | — | — | — | — | 440 mg | 3 ml |
| P11 | Compound III. | | | | | | — |
| P12 | 6.945 mg | — | — | — | 69.45 mg | — | 3 ml |
| P13 | 6.945 mg | — | — | — | — | 69.45 mg | 3 ml |

Furthermore, the animal CS, vegetable CS I and vegetable CS II ingredients were tested alone to compare them:

TABLE B

| TEST | INGREDIENTS | FINAL VOLUME |
|---|---|---|
| P14 | 440 mg of animal CS | 3 ml |
| P15 | 440 mg of vegetable CS I | 3 ml |
| P16 | 440 mg vegetable CS II | 3 ml |

### 4. Methods

### 4.1 Caffeine Permeability Test

For percutaneous absorption studies, OECD guideline 428 recommends the use of caffeine (MW=194.2 log P o/w −0.08) as a low lipophilic reference compound. This molecule has been used in various studies to evaluate permeability and therefore the use of different models of epidermis reconstructed in vitro for percutaneous absorption studies. As regards the ability to overcome the epithelial barrier even in the absence of damage, caffeine was used as a probe to evaluate the propensity of a product to form a protective film: the reduction of the passage of caffeine through the biological model is then used as an index of film-forming property.

### Procedure

After 15 minutes of treatment with the test element, 100 μl of 0.5% caffeine solution at pH 3.3 were applied in the apical compartment (donor) for a 2-hour exposure.

After 15 minutes, 1 hour and 2 hours, a sample (1 ml) was collected from the receptor fluid and stored at 2-8° C. for UPLC analysis.

### Data Acquisition and Analysis

The caffeine concentration in the 0.5% reference solution and collected biological samples was quantified using the UPLC method by an external analytical laboratory (LabAnalysis Srl). The results are expressed in μg and % of caffeine spread with respect to the dose applied, as well as with respect to the negative control.

### 4.2 Lucifer Yellow Assay

Lucifer Yellow (in short LY) is a fluorescent dye impermeable to the cell membrane and it is used as a probe to study paracellular permeability in tissues. In cases where cell junctions are uninterrupted, lucifer yellow has a very low permeability; however, if the joints between the cells are damaged, the diffusion of lucifer yellow increases. In this case, the dye was used to verify the integrity of the cell junctions in the tissues in the presence of the test product.

### Procedure

After treatment, the test product was removed and 0.5 mL of lucifer yellow (500 μM in saline solution) was applied to the apical compartment (in the insert) of the tissue. The saline solution (1 mL) was added to the basolateral compartment and LY was allowed to incubate for a period of 1 hour at 37° C. The diffusion of LY was evaluated by quantifying changes in fluorescence levels between the apical and basolateral compartment of the tissue.

### Data Acquisition

The fluorescent reading was carried out in the spectrofluorometer (TECAN INFINITE M200) with excitation of 428 nm and emission wavelengths of 535 nm.

For each tissue, fluorescence measurement (RFU) was carried out at basolateral level and LY flow was calculated based on the following formula:

LY Flux %=(RFU BL/RFU AP t=0)×100

(BL=basolateral; AP t=0=apical; RFU mean of LY 500 μM).

### 5. Results

### 5.1 Caffeine Permeability Test

In Table C (columns 2-4) the results are expressed as a percentage of reduction in caffeine passage (i.e., film-forming efficacy) considering as 100% the dose quantified in the negative control (53.5 μg, 115.6 μg and 114.2 μg after 15 minutes, 1 hour and 2 hours, respectively): negative controls are considered to be 0% of film-forming efficacy.

Globally, combinations comprising a chondroitin sulfate of plant origin according to the invention showed a film-forming efficacy.

### 5.2. Lucifer Yellow Assay of Paracellular Permeability

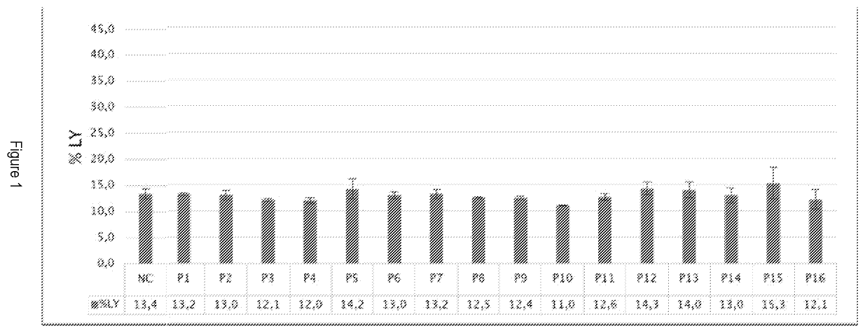

The paracellular flow of the Lucifer Yellow dye (LY) was evaluated at the end of the exposure time (15 minutes+2 hours), and fluorescence measurements are reported in FIG. 1 and in Table C (column 5). The negative control had a permeability to Lucifer yellow of 13.4%.

Permeability to Lucifer yellow recorded after 15 minutes+2 hours of treatment with all the compounds under analysis, alone or combined, was not modified with respect to the negative and positive controls.

For technical reasons the white petroleum jelly series (film-forming or positive control reference) did not give the expected results and cannot be considered a valid reference: no significant decrease in caffeine passage was observed and the LY % flow increased significantly.

The epithelial permeability measured by the Lucifer yellow assay showed that no changes occurred during exposure for all tested compounds with respect to the unexposed negative control (treated with acidic caffeine pH 3.3), confirming the maintenance of barrier integrity during the experimental period.

### 5.3. Measuring the pH

After 2 hours from the application of the caffeine and after the collection of the receptor fluid, the pH of the apical compartment was measured (with litmus paper having a precision range of 2.9-6.5). The results are reported in Table C (column 6).

Most of the compounds tested showed buffer properties with respect to the negative control (pH 6.5), in particular the mixtures or the compositions according to the invention P3, P4, P6, and P7.

Table C Anova T-test post-hoc Tukey: p < 0.05(*), p < 0.01(**)

| | | | | Barrier | |
|---|---|---|---|---|---|
| | | | | integrity: | Measurement |
| | Film forming efficacy | | | end of study | at the end |
| FILM | % decrease in caffeine passage compared to negative control | | | Lucifer | of the study |
| FORMING | 15 minutes | 1 hour | 2 hours | Yellow % | pH |
| NC | 0.1 ± 0.93 | 0.0 ± 1.71 | 0.0 ± 1.76 | 13.4 | 6.5 ± 0.0 |
| PETROLEUM | 35.4 ± 3.40 (**) | 39.7 ± 1.63 (**) | 18.5 ± 2.23 (**) | 39.6 | 6.5 ± 0.0 |
| JELLY | | | | | |
| P1 | 67.8 ± 5.56 (**) | 46.1 ± 2.18 (**) | 31.4 ± 1.30 (**) | 13.2 | 8 ± 0.0 |
| P2 | 60.7 ± 1.90 (**) | 37.2 ± 1.76 (**) | 24.8 ± 4.26 (**) | 13.0 | 7 ± 0.0 |
| P3 | 53.2 ± 2.25 (**) | 35.8 ± 0.99 (**) | 20.4 ± 2.24 (**) | 12.1 | 7 ± 0.0 |
| P4 | 70.8 ± 2.85 (**) | 38.5 ± 1.23 (**) | 17.1 ± 3.46 (**) | 12.0 | 7 ± 0.0 |
| P5 | 91.7 ± 3.51 (**) | 64.9 ± 3.69 (**) | 40.0 ± 3.02 (**) | 14.2 | 8 ± 0.0 |
| P6 | 82.7 ± 5.24 (**) | 52.7 ± 2.64 (**) | 25.2 ± 1.62 (**) | 13.0 | 8 ± 0.0 |
| P7 | 84.3 ± 5.28 (**) | 44.8 ± 5.5 (**) | 20.3 ± 3.22 (**) | 13.2 | 8 ± 0.0 |
| P8 | 69.7 ± 1.19 (**) | 37.1 ± 2.05 (**) | 28.1 ± 0.78 (**) | 12.5 | 6.5 ± 0.0 |
| P9 | 62.4 ± 1.94 (**) | 40.4 ± 3.94 (**) | 14.3 ± 1.16 (**) | 12.4 | 6.5 ± 0.0 |
| P10 | 67.0 ± 5.97 (**) | 36.6 ± 3.19 (**) | 18.9 ± 1.82 (**) | 11.0 | 6 ± 0.0 |
| P11 | 85.2 ± 3.29 (**) | 56.1 ± 2.51 (**) | 19.2 ± 2.09 (**) | 12.6 | 7 ± 0.0 |
| P12 | 43.0 ± 9.50 (**) | 12.7 ± 3.24 (*) | 8.9 ± 0.06 (**) | 14.3 | 7 ± 0.0 |
| P13 | 28.6 ± 14.04 (**) | 9.3 ± 3.04 | 4.9 ± 1.01 (*) | 14.0 | 7 ± 0.0 |
| P14 | 59.6 ± 2.12 (**) | 33.9 ± 0.76 (**) | 25.6 ± 0.91 (**) | 13.0 | 6.5 ± 0.0 |
| P15 | 26.1 ± 1.12 (**) | 33.2 ± 1.36 (**) | −2.3 ± 2.28 | 15.3 | 6.5 ± 0.0 |
| P16 | 64.4 ± 7.47 (**) | 36.6 ± 7.51 (**) | 15.4 ± 1.66 (**) | 12.1 | 6.5 ± 0.0 |

Embodiments E(n) of the present invention are illustrated below:

E1. A composition comprising:
a mixture comprising, a or alternatively, consisting of:
   a vegetable substance comprising or, alternatively, consisting of an extract of at least one alga, wherein said at least one alga comprises or, alternatively, consists of an alga belonging to the species Ulva lactuca (L.) and/or an alga belonging to the genus Fucus (L.), and
   a hyaluronic acid or a salt thereof, wherein said hyaluronic acid has an average molecular weight comprised from 200 kDa to 1500 kDa;
and wherein said composition further comprises at least one food or pharmaceutical grade additive and/or excipient.

E2. The composition according to E1, wherein said mixture further comprises a basic substance, wherein said basic substance is selected from a group comprising or, alternatively, consisting of aluminium hydroxide, magnesium hydroxide, magnesium trisilicate, sodium hydroxide and a mixture thereof; preferably aluminium hydroxide or magnesium trisilicate.

E3. The composition according to E1 or E2, wherein said extract of at least one alga comprises or, alternatively, consists of an extract of said alga belonging to the species Ulva lactuca and of said alga belonging to the genus Fucus, wherein said alga belonging to the genus Fucus is an alga belonging to the species Fucus vesiculosus.

E4. The composition according to any one of E1-E3, wherein said composition is in solid form for oral use of tablet as such or tablet to be chewed or tablet to be dissolved in the mouth;

preferably, wherein said mixture comprises or, alternatively, consists of:
said vegetable substance comprising or, alternatively, consisting of: an extract of Ulva lactuca and Fucus vesiculosus and, optionally, a hyaluronic acid or a salt thereof, and
said further hyaluronic acid or a salt thereof, wherein said hyaluronic acid has an average molecular weight comprised from 200 kDa to 1500 kDa, preferably a sodium hyaluronate, and
said aluminium hydroxide or magnesium trisilicate.

E5. The composition according to any one of E2-E4, wherein said hyaluronic acid or a salt thereof (HA) and said basic substance and said vegetable substance are at a [HA:basic substance:vegetable substance] weight ratio comprised from 1:10:20 to 1:30:60, preferably from 1:15:30 to 1:25:50, more preferably about 1:20:40.

E6. The composition according to any one of E1-E3, wherein said composition is in liquid form for oral use;

preferably, wherein said mixture comprises or, alternatively, consists of:
a vegetable substance comprising or, alternatively, consisting of: an extract of Ulva lactuca and Fucus vesiculosus and, optionally, a hyaluronic acid or a salt thereof, and
a further hyaluronic acid or a salt thereof, wherein said hyaluronic acid has an average molecular weight comprised from 200 kDa to 1500 kDa, preferably sodium hyaluronate,

(d) honey, and

(e) an Aloe vera gel.

E7. The composition according to E6, wherein said composition is in form of a water-based liquid or hydroalcoholic-based liquid, preferably a water-based syrup or a hydroalcoholic-based syrup.

E8. The composition according to E6 or E7, wherein said hyaluronic acid or a salt thereof (HA) and said vegetable substance are at a [HA: vegetable substance] weight ratio comprised from 1:3 to 1:20, preferably from 1:5 to 1:15, more preferably about 1:10.

E9. The composition according to any one of E1-E8 for use as medicament.

E10. The composition according to any one of E1-E8 for use in a method for the preventive or curative treatment of a disease and/or symptom selected from the group comprising or, alternatively, consisting of:
gastroesophageal reflux disease (GERD), laryngopharyngeal reflux disease (LPR), lesions or ulcers in the mucosa or tissues of the oral cavity, lesions or ulcers in the mucosa or tissues of the pharyngolaryngeal tract, lesions or ulcers in the mucosa or tissues of the oesophageal tract, oesophageal ulcers, deepithelialization of the oesophageal mucosa, peptic ulcers, gastric ulcers;
mucositis, aphthous, aphthoid lesions;
acute or chronic inflammations of the mucous membrane or tissues of the oral cavity, acute or chronic inflammations of the mucous membrane or tissues of the pharyngolaryngeal tract, acute or chronic inflammations of the mucous membrane or tissues of the oesophageal tract, oesophagitis or acute or chronic inflammation of the oesophageal mucosa;
acid regurgitation, heartburn, feeling of gastric fullness, epigastric pain, dyspepsia, nausea, chronic cough, bronchospasm, inflamed throat, laryngitis, globus sensation or hypopharyngeal bolus, pyrosis, dysphonia, nasopharyngeal inflammation, laryngospasm, raucousness, excessive throat clearing, throat pain or burning, postnasal drip, pharyngitis and/or other symptoms of the upper respiratory tract.

Further embodiments F(n) of the present invention are illustrated below:

F1. A composition in solid form for oral use of tablet as such or tablet to be chewed or tablet to be dissolved in the mouth, wherein said composition comprises:
a mixture comprising, a or alternatively, consisting of:
   a vegetable substance comprising or, alternatively, consisting of an extract of at least one alga, wherein said at least one alga comprises or, alternatively, consists of an alga belonging to the species Ulva lactuca (L.) and/or an alga belonging to the genus Fucus (L.), and
   a hyaluronic acid or a salt thereof, wherein said hyaluronic acid has an average molecular weight comprised from 200 kDa to 1500 kDa;
and wherein said composition further comprises at least one food or pharmaceutical grade additive and/or excipient.

F2. The composition according to F1, wherein said mixture further comprises a basic substance, wherein said basic substance is selected from a group comprising or, alternatively, consisting of aluminium hydroxide, magnesium hydroxide, magnesium trisilicate, sodium hydroxide and a mixture thereof; preferably aluminium hydroxide or magnesium trisilicate.

F3. The composition according to F1 or F2, wherein said extract of at least one alga comprises or, alternatively, consists of an extract of said alga belonging to the species Ulva lactuca and of said alga belonging to the genus Fucus.

F4. The composition according to any one of F1-F3, wherein said alga belonging to the genus Fucus is an alga belonging to the species Fucus vesiculosus.

F5. The composition according to any one of F1-F4, wherein said mixture comprises or, alternatively, consists of:
said vegetable substance comprising or, alternatively, consisting of: an extract of Ulva lactuca and Fucus vesiculosus and, optionally, a hyaluronic acid or a salt thereof, and
said further hyaluronic acid or a salt thereof, wherein said hyaluronic acid has an average molecular weight comprised from 200 kDa to 1500 kDa, and
said aluminium hydroxide or magnesium trisilicate.

F6. The composition according to F5, wherein said further hyaluronic acid or a salt thereof is a sodium hyaluronate.

F7. The composition according to any one of F2-F6, wherein said hyaluronic acid or a salt thereof (HA) and said basic substance and said vegetable substance are at a [HA:basic substance:vegetable substance] weight ratio comprised from 1:10:20 to 1:30:60, preferably from 1:15:30 to 1:25:50, more preferably about 1:20:40.

F8. The composition according to any one of F1-F7 for use as medicament.

F9. The composition according to any one of F1-F7 for use in a method for the preventive or curative treatment of a disease and/or symptom selected from the group comprising or, alternatively, consisting of:
gastroesophageal reflux disease (GERD), laryngopharyngeal reflux disease (LPR), lesions or ulcers in the mucosa or tissues of the oral cavity, lesions or ulcers in the mucosa or tissues of the pharyngolaryngeal tract, lesions or ulcers in the mucosa or tissues of the oesophageal tract, oesophageal ulcers, deepithelialization of the oesophageal mucosa, peptic ulcers, gastric ulcers;
mucositis, aphthous, aphthoid lesions;
acute or chronic inflammations of the mucous membrane or tissues of the oral cavity, acute or chronic inflammations of the mucous membrane or tissues of the pharyngolaryngeal tract, acute or chronic inflammations of the mucous membrane or tissues of the oesophageal tract, oesophagitis or acute or chronic inflammation of the oesophageal mucosa;
acid regurgitation, heartburn, feeling of gastric fullness, epigastric pain, dyspepsia, nausea, chronic cough, bronchospasm, inflamed throat, laryngitis, globus sensation or hypopharyngeal bolus, pyrosis, dysphonia, nasopharyngeal inflammation, laryngospasm, raucousness, excessive throat clearing, throat pain or burning, postnasal drip, pharyngitis and/or other symptoms of the upper respiratory tract.

1. A composition for oral use, wherein said composition comprises:
a mixture comprising:
a vegetable substance comprising an extract of at least one alga, wherein said at least one alga comprises an alga belonging to the species Ulva lactuca (L.) and/or an alga belonging to the genus Fucus (L.), and
a hyaluronic acid or a salt thereof, wherein said hyaluronic acid has an average molecular weight comprised from 200 kDa to 1500 kDa;
and wherein said composition further comprises at least one food or pharmaceutical grade additive and/or excipient.

2. The composition according to claim 1, wherein said mixture further comprises a basic substance, wherein said basic substance is selected from the group consisting of aluminium hydroxide, magnesium hydroxide, magnesium trisilicate, sodium hydroxide and a mixture thereof.

3. The composition according to claim 1, wherein said extract of at least one alga comprises an extract of said alga belonging to the species Ulva lactuca and of said alga belonging to the genus Fucus.

4. The composition according to claim 1, wherein said alga belonging to the genus Fucus is an alga belonging to the species Fucus vesiculosus.

5. The composition according to claim 1, wherein said mixture comprises:
said vegetable substance comprising: an extract of Ulva lactuca and Fucus vesiculosus and, optionally, a hyaluronic acid or a salt thereof, and
said further hyaluronic acid or a salt thereof, wherein said hyaluronic acid has an average molecular weight comprised from 200 kDa to 1500 kDa, and
said sodium hydroxide, aluminium hydroxide or magnesium trisilicate.

6. The composition according to claim 5, wherein said further hyaluronic acid or a salt thereof is a sodium hyaluronate.

7. The composition according to claim 2, wherein said hyaluronic acid or a salt thereof (HA) and said basic substance and said vegetable substance are at a [HA:basic substance: vegetable substance] weight ratio comprised from 1:10:20 to 1:30:60.

8. The composition according to claim 1 for use as medicament.

9. Method of preventing or treating a subject in need thereof a disease and/or symptom with the composition according to claim 1, said disease or symptom being selected from the group consisting of:
gastroesophageal reflux disease (GERD), laryngopharyngeal reflux disease (LPR), lesions or ulcers in the mucosa or tissues of the oral cavity, lesions or ulcers in the mucosa or tissues of the pharyngolaryngeal tract, lesions or ulcers in the mucosa or tissues of the oesophageal tract, oesophageal ulcers, deepithelialization of the oesophageal mucosa, peptic ulcers, gastric ulcers;
mucositis, aphthous, aphthoid lesions;
acute or chronic inflammations of the mucous membrane or tissues of the oral cavity, acute or chronic inflammations of the mucous membrane or tissues of the pharyngolaryngeal tract, acute or chronic inflammations of the mucous membrane or tissues of the oesophageal tract, oesophagitis or acute or chronic inflammation of the oesophageal mucosa;
acid regurgitation, heartburn, feeling of gastric fullness, epigastric pain, dyspepsia, nausea, chronic cough, bronchospasm, inflamed throat, laryngitis, globus sensation or hypopharyngeal bolus, pyrosis, dysphonia, nasopharyngeal inflammation, laryngospasm, raucousness, excessive throat clearing, throat pain or burning, postnasal drip, pharyngitis and/or other symptoms of the upper respiratory tract.

10. The composition according to claim 2, wherein said basic substance is selected from the group consisting of aluminum hydroxide and magnesium trisilicate.

11. The composition according to claim 7, wherein the weight ratio HA: basic substance: vegetable substance is 1:15:30 to 1:25:50.

12. The composition according to claim 7, wherein the weight ratio HA: basic substance: vegetable substance is 1:20:40.

13. The composition according to claim 5 in solid form for oral use, wherein said mixture comprises:
said vegetable substance comprising: an extract of Ulva lactuca and Fucus vesiculosus and, optionally, a hyaluronic acid or a salt thereof, and
said further hyaluronic acid or a salt thereof, wherein said hyaluronic acid has an average molecular weight comprised from 200 kDa to 1500 kDa, and
said aluminium hydroxide or magnesium trisilicate.

14. The composition according to claim 5 in liquid form for oral use, wherein said mixture comprises:
said vegetable substance comprising: an extract of Ulva lactuca and Fucus vesiculosus and, optionally, a hyaluronic acid or a salt thereof, and
said further hyaluronic acid or a salt thereof, wherein said hyaluronic acid has an average molecular weight comprised from 200 kDa to 1500 kDa, and
a honey, and
an Aloe vera gel.
